(19) United States
(12) Patent Application Publication
(10) Pub. No.: US 2025/0020734 A1
(43) Pub. Date: Jan. 16, 2025

(54) Continuity Clip

(71) Applicant: Vishnu Vardhan Chakravaram, Gallatin, TN (US)

(72) Inventor: Vishnu Vardhan Chakravaram, Gallatin, TN (US)

(73) Assignee: Vishnu Vardhan Chakravaram, Gallatin, TN (US)

(21) Appl. No.: 18/734,837

(22) Filed: Jun. 5, 2024

Related U.S. Application Data
(63) Continuation of application No. 63/457,475, filed on Apr. 6, 2023.

(51) Int. Cl. G01R 31/54

(52) U.S. Cl. CPC G01R 31/54

(57) ABSTRACT

The continuity clip assembly arranged with either a magnet or suction cup and a probe holder, becomes helpful apparatus to check continuity of a harness or bundle of wires. The continuity clip assembly can be fitted with magnet under its base, which ensures clips stability when placed on metal surface. It can be fitted with a suction cup under its base, which ensures clip's stability when continuity clip assembly is placed on a smooth surface. The continuity clip assembly can be fitted with a probe holder on the side which can be used to hold the probe of multimeter over the desired pins or position. The probe holder assembly can be fitted with either a magnet or suction cup. The prove holder assembly comes to rescue by holding a probe at a specific pin location while the harness is assembled on the appliance eliminating the need for holding the probe with hands.

### CROSS-REFERENCE TO RELATED APPLICATIONS

This application claims the benefit of provisional application No. 63/457,475, filed on Apr. 6, 2023.

### BACKGROUND OF THE INVENTION

To check continuity between two pins on two different connectors on a harness or just wires going from one point to another point, a man must have four hands, two of them to hold the harness steady so the connectors are in an ideal position and the other two hands to hold the probes at desired connector pins. However, the problem is, here on earth a man has only two hands. Hence the need for a device to hold the harness in place so the two-handed man can check the continuity with ease by just poking probes into the desired connector pins.

### BRIEF SUMMARY OF THE INVENTION

In one embodiment the composite structure is a continuity clip, wherein the continuity clip is fitted with a magnet on the bottom side of the base of dual clip and this embodiment is used to stick the composite structure to a metal surface. In another embodiment, the composite structure is a continuity clip, wherein the continuity clip is fitted with a suction cup on the bottom side of the base of dual clip and this embodiment is used to stick the composite structure to a plane smooth surface. In another embodiment, the composite structure is a continuity clip, wherein the composite structure is fitted with either a magnet or suction cup and a probe holder.

In another embodiment just the probe holder attached to a base of dual clip which can be fitted with a magnet or a suction cup, can be used to hold the probe of a multimeter at a desired connectors pin location. In another embodiment just the probe holder attached to a base of dual clip which can be fitted with a magnet or a suction cup, can be used to hold the probe of a multimeter at a desired connector's pin location.

To illustrate the versatility the composite structure can be installed on a surface that is either smooth or made of metal. Smooth surface such as glass, so that the suction cup can make a firm contact and a metal surface so the magnet can make a from contact. If the user plans to use the composite structure at a work bench that is neither metal nor smooth surface, then an L bracket is used to elevate the composite structure and hold on the table at the same time. The installed composite structure can be used to bite on to a bundle wire with connectors, so the user can effortlessly check the continuity between two pins of different or same connectors.

To illustrate the versatility the composite structure can be installed on a surface, where there is a need to hold the harness or bundle of similar things firmly at a position or where there is a need to hold the probes of multimeter on a connector at a certain pin location. The composite structure can be configured for myriad of usages. For example, the composite structure can be used to hold a bundle of wires and hold multimeter probes at a certain desired pin location on a chosen connector.

The various embodiments enable, support and/or provide a new paradigm in the field of electrical & mechanical tools. The embodiments disclose a continuity clip, which enables the user with an easy and effortless act of checking continuity between two points. This embodiment of continuity clip provides a great solution for checking continuity without the need to hold the wires and probes at the same time. For example, use plugs the wire harness/bundle of wires to the continuity clip and effortlessly checks the continuity.

### BRIEF DESCRIPTION OF THE SEVERAL VIEWS OF THE DRAWING(S)

In order that the invention more fully understood, it will now be described, by way of example, with reference to the accompanying drawing in which:

FIG. 1A is an illustrative isometric view of continuity clip assembly of the present invention in use;

FIG. 1B is an illustrative side view of continuity clip assembly of the present invention in use;

FIG. 2 is an illustrative isometric view of base of dual clip of the present invention;

FIG. 3 is an illustrative isometric view of wing clip of the present invention;

FIG. 4 is an illustrative isometric view of continuity clip assembly with magnet and probe holder assembly of the present invention in use;

FIG. 5 is an illustrative isometric view of continuity clip assembly with suction cup and probe holder assembly of the present invention in use;

FIG. 6 is an illustrative isometric view of probe holder assembly that includes magnet of the present invention in use;

FIG. 7 is an illustrative isometric view of probe holder assembly that includes suction cup of the present invention in use;

FIG. 8 is an illustrative top view of horizontal U-clamp assembly of present invention in use;

FIG. 9 is an illustrative top view of horizontal slider assembly of the present invention in use;

FIG. 10 is an illustrative isometric view of parent and child bolt assembly of the present invention in use;

FIG. 11A is an illustrative isometric view of continuity clip assembly, multimeter with probes, connectors, probe assembly and Vertical and horizontal clamp assembly, attached to a tabletop of the present invention in use;

FIG. 11B is an illustrative side view of continuity clip assembly, multimeter with probes, connectors, probe assembly and Vertical and horizontal clamp assembly, attached to a tabletop of the present invention in use;

FIG. 12 is an illustrative isometric view of U-bolt of the present invention in use;

FIG. 13 is an illustrative isometric view of half slider of the present invention in use;

FIG. 14 is an illustrative isometric view of bolt with stopper of the present invention in use;

### DETAILED DESCRIPTION OF THE INVENTION

The invention will be primarily described within the context of embodiments; however, those skilled in the art and informed by the teachings herein will realize that the invention is also applicable to other technical areas and/or embodiments. Practitioners skilled in the art will recognize numerous other embodiments as well.

The illustrative embodiments described herein are not meant to be limiting. It may be readily understood that certain aspects of the disclosed embodiments can be arranged and combined in a variety of different configurations, all of which are contemplated herein.

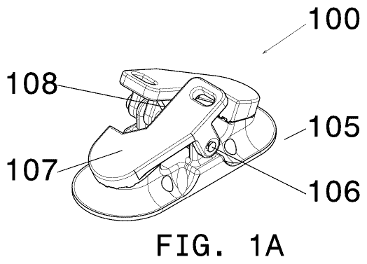

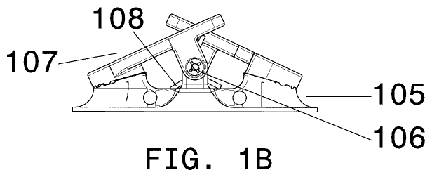

FIG. 1A-14 illustrates a composite structure 100, 400, 500, 600, 700, 800, 900, 1000, 1100.

FIG. 1A depicts isometric view of an exemplary composite structure according to an embodiment. The composite structure preferably consists of 4 major segments namely, (1) base of dual clip 105, (2) wing clip 107, (3) parent bolt 106, (4) spring 108. The composite structure 100, can be primarily used to hold on to any object preferably electrical wire harness or bundle of wires. User press down wing clip 107 on either sides of base of dual clip 105 to insert any object that user prefers to be held by the composite structure.

FIG. 1B depicts side view of an exemplary composite structure according to an embodiment. The composite structure preferably consists of 4 major segments (1) base of dual clip, (2) wing clip, (3) parent bolt, (4) spring 108.

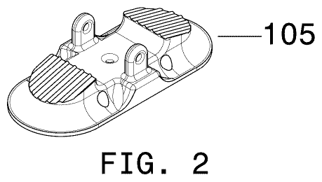

FIG. 2 depicts isometric view of an exemplary base of dual clip 105, according to the embodiment of the composite structure of FIG. 1.

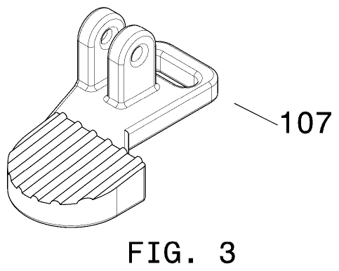

FIG. 3 depicts isometric view of an exemplary wing clip 107, according to the embodiment of the composite structure of FIG. 1.

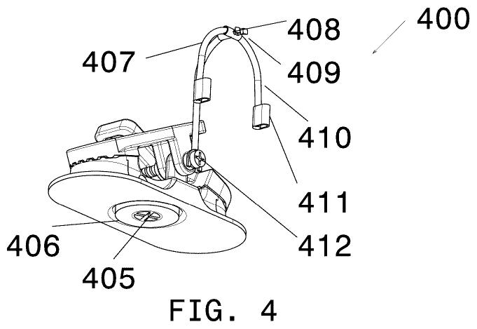

FIG. 4 depicts isometric view of an exemplary composite structure according to an embodiment. The composite structure preferably consists of 12 segments namely (1) base of dual clip 105, (2) wing clip 107, (3) parent bolt 106, (4) spring 108, (5) magnet 406, (6) screw 405, (7) child bolt 412, (8) probe arm primary 407, (9) T-joint 408, (10) wing screw 409, (11) probe arm secondary 410, (12) Velcro 411. As described above composite structure 100 can be assembled with magnet 406, probe arm primary 407 & probe arm secondary 412. Magnet 406 allows the composite structure 100 to sit stable on the surface without tripping. Probe primary arm 407 & Probe secondary arm 410 allows the composite structure to hold probes at a fixed position/location.

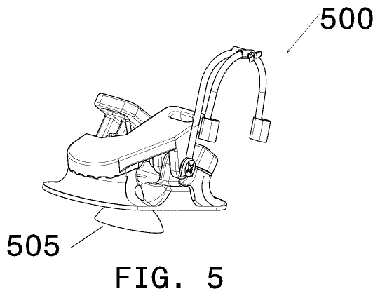

FIG. 5 depicts isometric view of an exemplary composite structure according to an embodiment the composite structure preferably consists of 12 segments namely (1) base of dual clip 105, (2) wing clip 107, (3) parent bolt 106, (4) spring 108, (5) suction cup 505, (6) screw 405, (7) child bolt 412, (8) probe arm primary 407, (9) T-joint 408, (10) wing screw 409, (11) probe arm secondary 410, (12) Velcro 411. As described above composite structure 100 can be assembled with magnet 406, probe arm primary 407 & probe arm secondary 412. suction cup 505 allows the composite structure 100 to sit stable on the surface without tripping. Probe primary arm 407 & Probe secondary arm 410 allows the composite structure to hold probes at a fixed position/location.

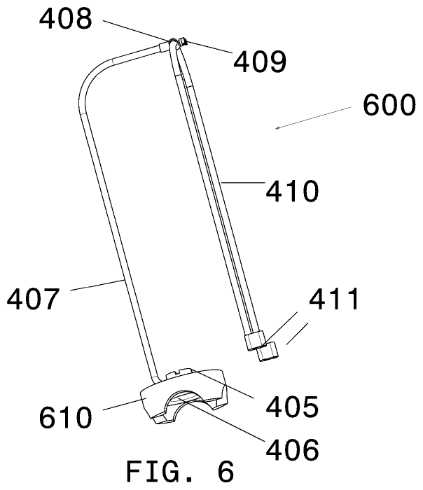

FIG. 6 depicts isometric view of an exemplary composite structure according to an embodiment. The composite structure preferably consists of 8 major segments namely, (1) fastener 405, (2) magnet 406, (3) probe arm primary 407, (4) T-joint 408, (5) wing screw 409, (6) probe arm secondary 410, (7) Velcro, (8) magnet holder 610. As described above composite structure 600 serves as a device to hold probes of multimeter at a desired connector's pin location. This helps the user check continuity of the system without the need to hold multimeter probes and wire harness in place. Magnet 406 ensures that the composite structure 600 is held firmly to the metal surface while the probe arm secondary and its Velcro 411 holds the multimeter probes.

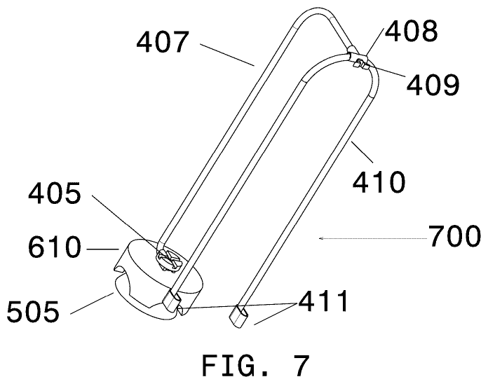

FIG. 7 depicts isometric view of an exemplary composite structure according to an embodiment. The composite structure preferably consists of 7 major segments namely, (1) fastener 405, (2) suction cup 505, (3) probe arm primary 407, (4) probe Velcro primary 408, (5) probe arm secondary 409, (6) probe Velcro secondary 410, (7) magnet holder 610. As described above composite structure 700 serves as a device to hold probes of multimeter at a desired connector's pin location. This helps the user check continuity of the system without the need to hold multimeter probes and wire harness in place. Suction cup 505 ensures that the composite structure 700 is held firmly to the metal surface while the probe arm secondary and its Velcro 411 holds the multimeter probes.

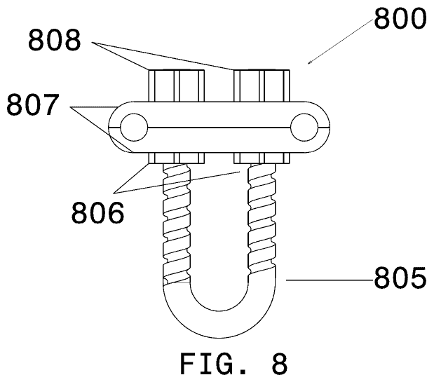

FIG. 8 depicts top view of an exemplary composite structure according to an embodiment. The composite structure preferably consists of 4 major segments namely, (1) U-bolt 805, (2) stopper nut 806, (3) half slider 807, (4) end nut 808. The composite structure 800 is used to hold on to the table or a surface. U-bolt 805 can be adjusted in position.

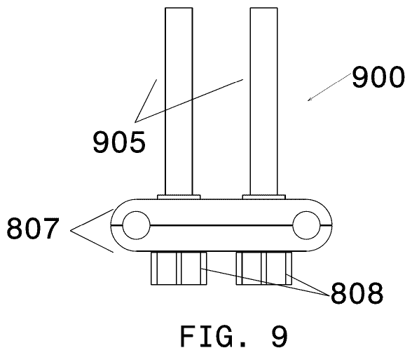

FIG. 9 depicts top view of an exemplary composite structure according to an embodiment. The composite structure preferably consists of 3 major segments namely, (1) half slider 807, (2) end nut 808, (3) bolt with stopper 905.

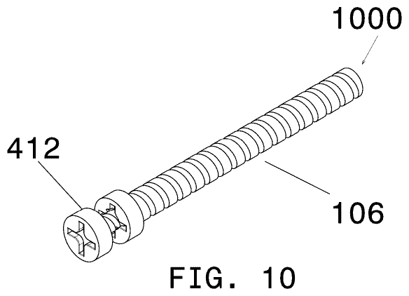

FIG. 10 depicts isometric view of an exemplary composite structure according to an embodiment. The composite structure preferably consists of 2 major segments namely, (1) parent bolt 106, (2) child bolt 412.

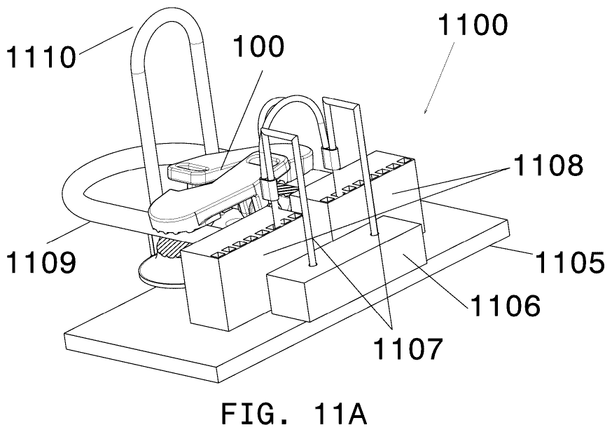

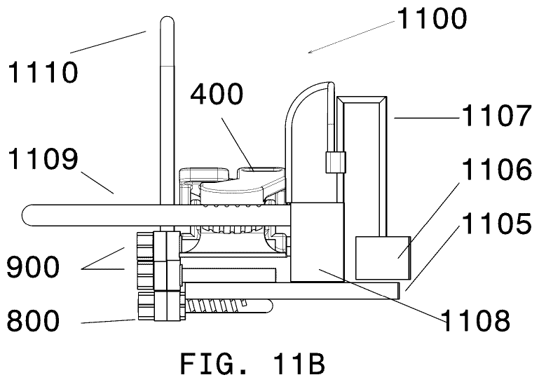

FIG. 11A depicts isometric view of an exemplary composite structure according to an embodiment. The composite structure preferably consists of 7 major segments namely, (1) continuity clip 100, (2) table 1105, (3) multimeter 1106, (4) multimeter probes 1107, (5) connector 1108, (6) wire harness 1109, (7) vertical U-clamp 1110.

FIG. 11B depicts side view of an exemplary composite structure according to an embodiment. The composite structure preferably consists of 9 major segments namely, (1) continuity clip 100, (2) table 1105, (3) multimeter 1106, (4) multimeter probes 1107, (5) connector 1108, (6) wire harness 1109, (7) vertical U-clamp 1110 (8) horizontal U-Clamp 800 (9) horizontal slider assembly 900.

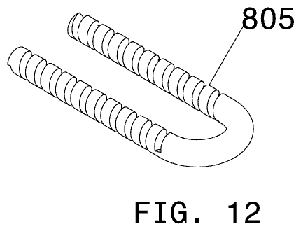

FIG. 12 depicts isometric view of an exemplary U-bolt 805, according to the embodiment of the composite structure of FIG. 1.

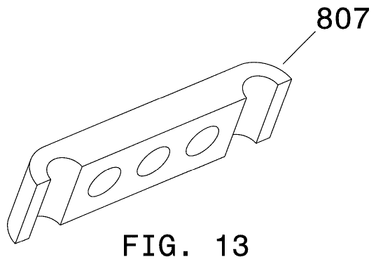

FIG. 13 depicts isometric view of an exemplary half slider 807, according to the embodiment of the composite structure of FIG. 1.

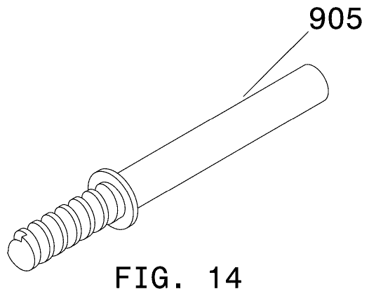

FIG. 14 depicts isometric view of an exemplary bolt with stopper 905, according to the embodiment of the composite structure of FIG. 1.

1. A composite structure which is a continuity clip comprising:
A base of dual clip with elevated surfaces on either side;
Said base of dual clip has extended foot base on its bottom side to give base extra surface area and prevent it from tripping;
Said base of dual clip has three holes on its length that runs through the entire breadth of the base of dual clip;
Among the said three holes, enter holes have threads to allow a both to be driven into, holes on either side of center holes can accommodate shafts;
Said base of dual clip has through hole for screw on the bottom side;
Said base of dual clip has a cylindrical pocket on the bottom side of the base to accommodate magnet and can be affixed to base using a screw;
Said base of dual clip has two elevated structures on either sides, to hold two wing clips over same axis;
Said wing clips can make an angular moment and is held over an axis using a parent bolt;
Said parent bolt is shared between the said wing clips and is fitted with two springs under each said wing clip for clip action.

2. The composite structure as described in claim-1 wherein said continuity clip further can be assembled with said magnet fitted at the bottom of said base of dual clip, said magnet is fixed to said base of dual clip using a fastener.

3. The composite structure as described in claim-1 wherein said continuity clip further can be used to hold both ends of the harness.

4. The composite structure as described in claim-1 wherein said continuity clip further can be attached with a probe arm primary using a child bolt that can be screwed on the head of the said parent bolt.

5. The composite structure as described in claim-2 wherein said magnet can be replaced by a suction cup and can be attached to the base of dual clip using said fastener.

6. The composite structure as described in claim-2 wherein said continuity clip can be attached to a metal surface firmly, said composite structure shall not trip due to weight of the harness.

7. The composite structure as described in claim-3 wherein the said continuity clip can be used to check continuity without the need to hold the said harness wires using hands.

8. The composite structure as described in claim-4 wherein the said probe arm primary can be rotated at an angle either to the left or right of the composite structure.

9. The composite structure as described in claim-4 wherein the said parent bolt and said child bolt can be a hex, Philip or any other type or types.

10. The composite structure as described in claim-4 wherein the said child bolt prevents the need to unscrew the said parent bolt from the composite structure or in any other device and the said child bolt helps to hold anything to the parent probe.

11. The composite structure as described in claim-4 wherein the said probe arm primary can be attached to the probe base.

12. The composite structure as described in claim-11 where in the said probe arm primary can be attached with a T-joint.

13. The composite structure as described in claim-12 where in the said T-join can be fitted with probe arm secondary.

14. The composite structure as described in claim-11 wherein the said probe base can be fitted with either a magnet or a suction cup with the said screw.

15. The composite structure as described in claim-14 wherein the said probe holder, probe base, screw, suction cup/magnet becomes probe assembly.

16. The probe assembly as described in claim-15 wherein the said probe assembly can be used to hold the said probes of said multimeter and the said probes be placed on the pins of connectors.

17. The probe assembly as described in claim-16 wherein the said probe assembly is used when the harness is placed on the product.

18. The clamp assembly consists of a table, a clamp in the shape of letter I, and three sliders in three configurations that can slide vertically on the clamp.

19. Said sliders as described in claim-18, go above and below the surface of the table to firmly hold on to the said table.

20. Said one of the sliders as described in claim-18 goes underneath the surface of the table, can further be moved/adjusted horizontally depending on how much Horizontal depth is available.

21. Said slider as described in claim-18 can hold the said continuity clip at required elevation from the surface of the table, so that the said connector can rest on the table.

22. The composite structure as described in claim-20 wherein probe arm secondary shall slide through said probe T-joint.

23. The composite structure as described in claim-21 where in probe arm secondary can be locked at any place on the length of the probe arm secondary using a wing screw.

24. The composite structure as described in claim-1 where the said base of dual clip and wing clip shall have teeth to hold the wire harness without slipping.

25. The composite structure as described in claim-1 where the said continuity clip can rotate around Z-axis.

26. The composite structure as described in claim-13 where the probe arm secondary is fitted with Velcro on either side.

27. The composite structure as described in claim-26 where the Velcro is used to hold the probes.
